(19) United States
(12) Patent Application Publication
(10) Pub. No.: US 2023/0399572 A1
(43) Pub. Date: Dec. 14, 2023

(54) POLYOL TREATMENT OF HYDRODEOXYGENATION FEEDSTOCK

(71) Applicant: Renewable Energy Group, Inc., Ames, IA (US)

(72) Inventors: Martin Haverly, Ames, IA (US); Ramin Abhari, Bixby, OK (US); David A. Slade, Ames, IA (US)

(73) Assignee: Renewable Energy Group, Inc., Ames, IA (US)

(21) Appl. No.: 18/332,327

(22) Filed: Jun. 9, 2023

Related U.S. Application Data
(60) Provisional application No. 63/350,717, filed on Jun. 9, 2022.

(51) Int. Cl. C10G 3/00; C07C 67/03; C07C 1/213

(52) U.S. Cl. CPC C10G 3/50; C07C 67/03; C07C 1/213; C10G 2400/02; C10G 2400/04; C10G 2400/28; C10G 2400/22

(57) ABSTRACT

The present invention relates to an improved process for treating lipid feedstocks such as spent vegetable oils and low-value animal fats for biofuel production. In particular, the invention relates to methods for providing an advantageous feedstock for production of hydrocarbon biofuels via hydrodeoxygenation.

### CROSS-REFERENCE TO RELATED APPLICATION

The present application claims priority from U.S. Provisional Application No. 63/350,717 filed Jun. 9, 2022, which is incorporated by herein by reference in its entirety.

### FIELD OF THE INVENTION

The invention relates to an improved process for treating lipid feedstocks such as spent vegetable oils and low-value animal fats for biofuel production. In particular, the invention relates to methods for providing an advantageous feedstock for production of hydrocarbon biofuels via hydrodeoxygenation.

### BACKGROUND OF THE INVENTION

Renewable diesel (RD) is an isoparaffinic compression ignition fuel produced by hydroprocessing of fats and oils. The RD production process includes the hydrodeoxygenation (HDO) of lipid fatty acids and/or glycerides to paraffinic hydrocarbons. In the HDO reaction, the glycerol backbone of the glyceride molecules is converted to propane as the fatty acids are transformed to diesel range hydrocarbons. These methods have been disclosed in several references including U.S. Pat. Nos. 7,846,323, 7,968,757, 8,026,401, 8,558,042, and 10,246,658, the disclosures of which are incorporated herein by reference thereto.

Commercial production of RD began in 2007 and has grown to about 2 billion gallons per year since. In order to ensure feedstock availability, producers have been exploring the use of lower quality lipid feeds such as used cooking oils, trap greases, and palm sludge oil.

These lower quality feeds have a number of contaminants that detrimentally affect RD process plants. A contaminant category of particular concern is organic chlorine compounds (referred to as organochlorines herein). Conventional pretreatment methods (e.g. citric/phosphoric acid degumming and silica or bleaching clay adsorption) are not particularly effective in removing these compounds from the lipid.

As the fatty acid/glycerides react with hydrogen at HDO temperatures, pressures, and catalyst contact times to hydrodeoxygenate into hydrocarbons, water, and carbon oxides, the organochlorines undergo hydrodechlorination into hydrocarbons and hydrogen chloride (HCl). In the presence of water vapor and HCl, any vapor condensation (e.g. in the HDO effluent cooler or feed-effluent exchanger) can result in hydrochloric acid formation. Hydrochloric acid is a well-known cause of stress corrosion and cracking in stainless steel used in piping, vessels and other equipment used for the processing of RD and other materials.

The corrosion pathways caused by hydrogen chloride are not limited to aqueous hydrochloric acid. Since the gas-phase byproducts of HDO also include hydrogen sulfide (maintained to retain sulfide catalyst activity) and ammonia (formed by hydrodenitrogenation of organonitrogen components), ammonium chloride (NH₄Cl) salt deposit or precipitation provides another corrosion source. Corrosion due to ammonium bisulfide (NH₄HS) salt deposit or precipitation, a common problem in most hydrotreater systems, is enhanced with ammonium chloride co-precipitation. Corrosion in the HDO reactor system can cause damage to reactor internals, thermocouples, heat-effluent exchangers, and cold separator condenser. Costly steel alloy upgrades are required for various system equipment and piping in order to address the corrosion risk, significantly increasing the capital costs for RD plants.

The presence of organochlorines in lipids is somewhat surprising. After all, the conventional wisdom has been that organochlorines do not occur naturally in nature.

Nevertheless, unique organochlorines have been observed and characterized in certain vegetable oils. One such class of compounds is monochloropropanediols (MCPD), including 3-monochloropropane-1, 2-diol (3-MCPD) and 2-monochloroporapne-1,3-diol (2-MCPD), and their corresponding fatty acid esters. The highest level of MCPDs has been observed in palm oils where the bleaching and deodorizing conditions are typically more severe (e.g. higher temperatures than those used for other vegetable oils). The MCPDs are believed to be the product of the reaction between a chlorine donor and the lipid. The chlorine donor itself may be formed by thermal decomposition of smaller organochlorine molecules. Thus, the first step in the “chlorine chain” is the formation of these smaller organochlorine molecules.

One source of chlorine taken up in the plant is believed to be the inorganic fertilizer components such as potassium chloride and ammonium chloride. During growth of the plant, these chloride compounds are taken up by the plant and accumulated in the fruit, seed and other parts of the plant. At the same time, municipal water, which has been chlorinated, is used for processing. In Asia, where much of palm oil is grown, it is common to use iron chloride as a flocculent in water treatment. The organochlorine molecules thus seem to form endogenously in the plant during maturation. However, as noted in the previous paragraph, it is the higher temperature processing of the oil that forms a chlorine donor and thus moves it into the lipid structure; e.g. as MCPD fatty acid esters. A similar explanation can be provided for organochlorines in used cooking oil, where the high temperature frying operation forms a chlorine donor

In addition to MCPD esters, organochlorines may be incorporated into lipids as chlorinated fatty acids (CFA). CFA can be in the form of free fatty acids, monoglycerides, diglycerides, triglycerides, or fatty acid alkyl esters. Chlorinated wax esters and sphingolipids have also been reported in the literature, though these are less common.

Organochlorine contamination may also be in the form of synthetic adulterants. These may include pesticides (i.e. insecticides, herbicides, fumigants, and fungicides), disinfectants, polymers, polymer additives (plasticizers, flame-retardants, stabilizers), and industrial cleaners or degreasers. Analysis of commercially sourced brown grease has shown presence of chloroethane, chloroform, chloromethane, 1, 2-dichloroethylenes, tetrachloroethylene and trichloroethylene at ppm levels (Ward, P. M. L. Journal of Food Protection, 2012, 75 (4); 731-737).

Furthermore, chlorine can be incorporated into lipids as either MCPDs or CFAs through the reaction of chlorine-containing synthetic adulterants and lipids. This has been evidenced by elevated organochlorine levels in used cooking oils (UCO).

Regardless of source, organochlorines are hydrodechlorinated into HCl and consequent aqueous and solid phase byproducts that cause corrosion and other processing problems during RD production.

In order to support the growth of the RD industry, a diverse range of renewable feedstocks and hydrocarbon fuel coproducts is sought. These include use of not only low-value and waste lipids, but also sugars. Technologies for conversion of cellulose and hemicellulose to C5/C6 sugars are at various stages of development, thus providing a pathway for abundant biofuel feedstock. However, these feeds have not yet been employed in the RD production process.

Some references disclose esterification of free fatty acids (FFAs) with glycerol to produce glycerides. U.S. Pat. Nos. 7,087,771 and 8,088,183 describe such a glycerolysis step for reducing FFA content of feeds for base-catalyzed transesterification. However, these and other esterification references do no teach or suggest viability of the technology for removal of organochlorines, or contaminants in general.

Chinese Patent Publication CN1053685580A describes a method for removing organochlorines from waste or used cooking oil. The method involves heating the waste oil in an electric desalter in the presence of water, followed by addition of a dechlorinating agent. The dechlorinating agent is described as an organochlorine transforming agent (a nucleophile such as sodium methylate, sodium hydroxide, etc.), an organochlorine transfer agent (an ammonium salt such as quaternary ammonium hydroxide for transferring the chlorine species into the aqueous phase), and a polar solvent. However, addition of sodium methylate and similar strong bases is expected to result in formation of soaps (and loss in yield) during treatment of low-value and waste lipids that are characterized by relatively high FFA content.

There is thus a need for a treatment process that removes organochlorine compounds from the HDO reactor feeds. Additionally, there remains an unmet need for incorporation of sugar-based feeds for production of renewable hydrocarbon coproducts during HDO.

### BRIEF SUMMARY OF THE INVENTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

One aspect of the invention relates to a method for treatment of low-value and waste lipids-specifically fats, oils, and greases (FOG)—for conversion to hydrocarbons via hydrodeoxygenation. The method includes esterification in the presence of a polyol. It has surprisingly been observed that the esterification results in the removal of organochlorine contaminants in the FOG, without the addition of a catalyst (e.g. sodium methylate or other strong bases) as disclosed in the prior art.

The method includes combining the FOG feedstock with a polyol in an esterification reactor where it is subjected to conditions of high temperature and low pressure. In some embodiments, water is introduced and contacted with the feedstock at esterification temperatures before or during esterification. The esterification reactor effluent is optionally washed with water and subjected to a liquid-liquid phase separation where an esterified FOG light phase is separated from a heavy phase including the removed organochlorines. The esterified FOG light phase has an organochlorine concentration that is lower than the organochlorine concentration of the FOG feedstock. In some embodiments, the esterified FOG light phase undergoes HDO wherein the carbon chains of the fatty acid and polyol components of the esters are converted to hydrocarbons of similar chain lengths.

Other features and advantages of the present invention will become apparent to those skilled in the art from the following detailed description. It is to be understood, however, that the detailed description of the various embodiments and specific examples, while indicating preferred and other embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

These, as well as other objects and advantages of this invention, will be more completely understood and appreciated by referring to the following more detailed description of the presently preferred exemplary embodiments of the invention in conjunction with the accompanying drawings, of which:

FIG. 1 is a flow diagram of an embodiment of the present disclosure;

FIG. 2 is a reaction scheme illustrating chemical reactions associated with the present disclosure; and

FIG. 3 is a graph providing a summary of the experimental results form Example 1.

### DETAILED DESCRIPTION OF THE INVENTION

The apparatuses and methods disclosed in this document are described in detail by way of examples and with reference to the figures. Unless otherwise specified, like numbers in the figures indicate references to the same, similar, or corresponding elements throughout the figures. It will be appreciated that modifications to disclosed and described examples, arrangements, configurations, components, elements, apparatuses, methods, materials, etc. can be made and may be desired for a specific application. In this disclosure, any identification of specific shapes, materials, techniques, arrangements, etc. are either related to a specific example presented or are merely a general description of such a shape, material, technique, arrangement, etc. Identifications of specific details or examples are not intended to be, and should not be, construed as mandatory or limiting unless specifically designated as such. Selected examples of apparatuses and methods are hereinafter disclosed and described in detail with reference made to FIGURES.

The present technology treats FOG feedstock having an organochlorine concentration greater than about 10 wppm and free fatty acid (FFA) content greater than about 5 wt. % through an esterification reaction with a polyol.

In some embodiments, the FFA content of the FOG feedstock is greater than about 10 wt. %, In embodiments, the FFA content of the FOG feedstock is greater than 12 wt. %. Surprisingly, the removal of organochlorine contaminants has been found to be improved when the FFA content of the FOG feedstock is greater than 12 wt. %.

Organochlorine concentration, as defined herein, is the chlorine concentration in a FOG sample after the FOG sample has been thoroughly washed with water. In some embodiments, the FOG feedstock organochlorine concentration is greater than 15 wppm, greater than 20 wppm, greater than 25 wppm, greater than 30 wppm, greater than 35 wppm, greater than 40 wppm, greater than 45 wppm, greater than 50 wppm, greater than 60 wppm, greater than 70 wppm, greater than 80 wppm, greater than 90 wppm, greater than 100 wppm, or is in a range between any two of these values. For instance, the organochlorine content of the FOG feedstock is between 10 and 100 wppm. In embodiments, the FOG feedstock FFA content is between 5 wt. % and 75 wt. %. In embodiments, the FOG feedstock FFA content is between 10 wt. % and 75 wt. %, between than 15 wt. % and 75 wt. %, or between 20 wt. % and 75 wt. %.

In addition to organochlorine contaminants, the FOG feedstock also includes a range of other contaminants such as metals, phosphorus, and silicon. The metal contaminants include primarily sodium, potassium, magnesium, calcium, iron, and copper. Phosphorus is typically present in natural fats and oils as phospholipid molecules, including non-hydratable phospholipids that are ionically bound to divalent metals such as calcium. Silicon is typically present as organosilicon contaminants, such as polysiloxane anti-foam additives.

Depending on the amount of phosphorus present, the FOG feedstock may be subjected to a pretreatment step before the treatment method of the present technology. In some embodiments, a method such as the one disclosed in U.S. Pat. No. 9,404,064 is used to reduce the phosphorus content of the FOG feedstock to less than 20 wppm. In such pretreatment methods, the FOG feedstock is contacted with aqueous phosphoric or citric acid whereby the non-hydratable phospholipids are made hydratable and transferred to the water/oil interface for removal via centrifugation in a disc-stack centrifuge apparatus. In such pretreatment methods, the FOG feedstock may also be contacted with an absorbent filter media (such as diatomaceous earth or cellulose), an adsorbent filter media (such as amorphous silica or bleaching clay), or a combination of the two, to remove solid contaminants and further remove metals

Whether subjected to a pretreatment step or not, the FOG feedstock is directed to an esterification reactor system for conversion according to the present technology. A polyol is introduced to the esterification reactor along with the FOG feedstock. The polyol reactant is used to convert the free fatty acids to fatty acid esters including polyesters. Examples of the fatty acid polyesters include mono-, di-, and tri-ester products of the reaction between the polyol and free fatty acids. The esterification reactions also include inter-esterifications whereby the bound fatty acids of the FOG form new esters with the polyol reactant. Without being bound to any particular theory, it is believed that the fatty acids that are part of the FOG feedstock in the form of fatty acid esters of MCPD migrate to form new esters with the polyol, thus releasing the MCPD for removal via water wash phase separation. Contacting the FOG with water at esterification temperatures accelerates the desired transformation by promoting hydrolysis of the MCPD esters. The esterification occurs in the absence of a catalyst.

The polyol is typically a purified product with the general molecular formula (CHOH)ₙH₂ where n=3, 4, 5, or 6. A preferred polyol is glycerol (n=3). Other polyols for esterification according to the present technology include erythritol (n=4), xylitol (n=5), and sorbitol (n=6). The amount of polyol introduced to the esterification reactor depends on the FFA and organochlorine content of the FOG feedstock and the type of polyol reactant. For a polyol with n hydroxyl groups, the stoichiometric ratio of FFA-to-polyol may be defined as n moles of polyol per mole of FFA (assuming oleic acid molecular weight). For example, the stoichiometric ratio of FFA-to-polyol is 3:1 for glycerol and 6:1 for sorbitol. In embodiments, the amount of polyol introduced to the esterification reactor is from about 20% to about 400% of the stoichiometric FFA-to-polyol ratio.

The esterification reaction is conducted at a temperature of about 150 C to about 280 C, typically from about 180 C to about 250 C. The reaction is preferably conducted under conditions of agitation at sub-ambient pressure. Preferred operating pressures for esterification according to the present technology are less than 14 psia, less than 13 psia, less than 11 psia, less than 10 psia, less than 9 psia, less than 8 psia, less than 7 psia, less than 6 psia, less than 5 psia, less than 4 psia, less than 3 psia, less than 2 psia, less than 1 psia, less than 0.9 psia, less than 0.8 psia, less than 0.7 psia, less than 0.6 psia, less than 0.5 psia, less than, 0.4 psia, less than 0.3 psia, less than 0.2 psia, and less than 0.1 psia, or in a range between any two of these values. In a preferred embodiment, the esterification is conducted at a pressure between 0.1 psia and 7 psia. These sub-ambient pressure conditions are maintained through an esterification reactor vacuum system. This system could include a vacuum pump or an eductor system.

Under the aforementioned conditions of temperature and pressure, the water byproduct of the esterification reaction is evaporated. The reactor system is thus equipped with provisions for removing the water byproduct through the vacuum system during the course of the reaction. In addition to water, some of the organochlorine species in the FOG feedstock may also be vaporized and removed through the vacuum system.

The esterification reaction may be conducted in a batch or a continuous reactor system. A preferred batch esterification reactor includes provisions for heating such as a mechanical agitator and a jacket for circulation of a heat transfer fluid or steam condensate. The batch reaction time for completing the esterification is between 60 and 600 minutes, preferably between 90 and 500 minutes.

The continuous reactor systems for esterification comprise a plurality of stirred tank reactors in series. In some embodiments, the continuous reactor system includes from two to eight reactors in series. Each reactor in the system includes provisions for heating and agitation as previously described for the batch reactor embodiment. Additionally, each reactor in the system includes provisions for dosing the polyol and removing water vapor. In a continuous reactor embodiment, six reactors are arranged in order of increasing operating temperatures. Regardless of the number of reactors, the residence time through the continuous reactor system is between 90 minutes and 500 minutes, preferably between 180 and 360 minutes.

The esterification reactor effluent is subsequently subjected to a water washing and separation step. The amount of water used ranges from about 3% to about 30% of the esterification reactor effluent. The water may be contacted with the esterification reactor effluent in an agitated tank, a shear mixer, a static mixer, or similar devices used to provide good contact between immiscible liquid phases.

The wash water is then removed from the washed esterification reactor effluent. Any liquid-liquid separator, such as a settler, a decanter, a drum, or a centrifuge may be used for this purpose.

In some embodiments, the water washing and liquid-liquid separator may be part of a subsequent pretreatment process. Thus, the wash water used to contact the esterification reactor effluent may also optionally include a dilute acid (such as citric acid or phosphoric acid) and a caustic solution (such as sodium hydroxide).

The heavy phase from the liquid-liquid separator includes the water used for washing the esterification reactor effluent, any unconverted polyol, and the removed organochlorine compounds/byproducts.

The washed reactor effluent exits the liquid-liquid separator as a light phase. This esterification reactor light phase has an organochlorine concentration that is less than the FOG feedstock. In embodiments, the organochlorine concentration is less than 10 wppm, less than 9 wppm, less than 8 wppm, less than 7 wppm, less than 6 wppm, less than 5 wppm, less than 4 wppm, less than 3 wppm, less than 2 wppm, or less than 1 wppm. In some embodiments, the organochlorine concentration of the esterification reactor effluent after washing step is in the range given by any two of these values; for instance, between 1 and 10 wppm, or between 1 and 5 wppm.

Depending on the amount of polyol introduced to the esterification system (e.g. when in stoichiometric excess), a polyol liquid may be separated from the esterification reactor effluent without a water washing step. In some embodiments, this unwashed light phase from liquid-liquid separation of esterification product from polyol has the reduced organochlorine concentrations specified in the previous paragraph.

The light phase with reduced organochlorine content is suitable for hydrodeoxygenation (HDO), including for production of renewable diesel. Since the esters produced in the esterification reactor include reaction products of polyol and fatty acids, the HDO products include hydrocarbons corresponding to both the fatty acid and polyol carbon numbers. As such, an HDO feedstock including esters formed by a C3 polyol (i.e. glycerol) and C18 fatty acids (e.g. stearic and oleic acids) hydrodeoxygenates into products comprising propane and octadecane. Similarly, an HDO feedstock which includes esters formed by a C6 polyol (i.e. sorbitol) and C16 fatty acids (e.g. palmitic acid) converts into products that include hexane and hexadecane. As such, the esterification of the present technology not only removes detrimental organochlorines from FOG feedstocks, but also provides the ability to produce a broader range of bio-based hydrocarbon products than previously disclosed.

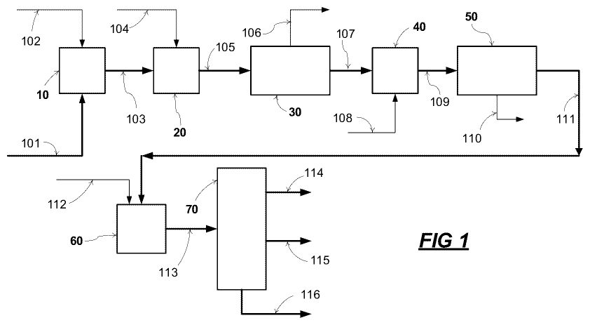

An embodiment of the present technology is provided in the FIG. 1 process flow block diagram. Referring to FIG. 1, a FOG feedstock 101 is contacted with a water feed 102 in an oil/water contactor 10. The FOG feedstock 101 has an organochlorine contaminant concentration between 10 wppm and 50 wppm and a FFA content greater than 5 wt. %. In preferred embodiments, the FFA content of the FOG feedstock 101 is greater than 12 wt. %. The water is preferably a demineralized water which is introduced at a rate between 3% and 10% of the FOG 101. The contactor 10 may be a static mixer or a stirred vessel. The contactor 10 is preferably operated at a temperature of about 150° C. to about 280° C., typically from about 180° C. to about 250° C. The contactor 10 pressure may range from atmospheric pressure to 1000 psig, preferably in the 0-100 psig range. Residence times in contactor 10 range from 2 to 60 minutes. At these conditions, at least some of the organochlorines are converted into water-soluble species; e.g. through hydrolysis of the MCPD esters. A water-contacted product 103, having higher FFA content than the FOG feed 101, leaves contactor 10 for mixing with a polyol feed 104 in polyol mixer 20.

The polyol feed 104 includes a polyol with the formula (CHOH)ₙH₂ where n=3, 4, 5, or 6. In embodiments, the polyol feed 104 includes sugar products xylitol and sorbitol. The amount of polyol introduced to the esterification reactor is about 30% to about 200% of the stoichiometric FFA-to-polyol ratio.

The temperature, pressure, and residence times for polyol mixer 20 are the same as those specified for the water contactor 10. The mixed stream 105 including polyols and FOG is processed through an esterification unit 30.

The esterification unit 30 has been described previously in this disclosure. In the present embodiment, the esterification unit 30 operates at a temperature of about 150° C. to about 280° C. (generally same as the water contactor 10 and the polyol mixer 30), typically from about 180° C. to about 250° C. The esterification unit 30 has two or more continuous stirred tank reactors (CSTRs) in series under a pressure between 0.1 psia and 7 psia. A water vapor stream 106 is withdrawn from the esterification unit 30.

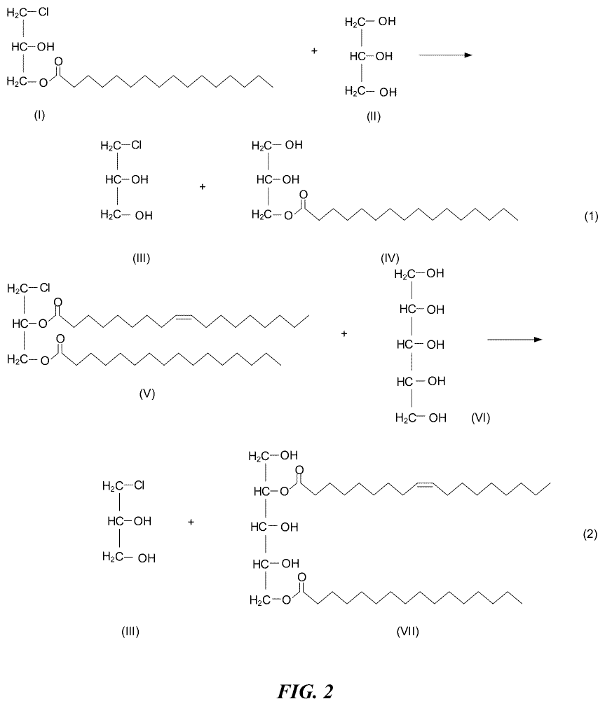

The water vapor removal promotes the desired esterification reaction between the polyols and the FFAs, while the additional residence time promotes the inter-esterification reactions that allow for redistribution of fatty acids among the MCPD, native glycerol (e.g. from FOG glycerides), and added polyols, thus releasing the MCPD from the water insoluble ester forms to the free MCPD form which is soluble in water. A schematic of potential inter-esterification reactions is shown in FIG. 2. Referring to FIG. 2, the palmitic acid ester of 3-MCPD (Structure I) and glycerol (Structure II) undergo inter-esterification according to Eq. 1 to form water-soluble 3-MCPD (Structure III) and a palmitic acid monoglyceride (Structure IV). Similarly, a 3-MCPD di-ester of oleic acid and palmitic acid (Structure V) may undergo inter-esterification with xylitol (Structure VI) according to Eq 2 to from the same water-soluble 3-MCPD and a fatty acid di-ester of xylitol (Structure VII).

Returning to FIG. 1, an esterification product 107 is washed with a wash-water 108 in a mixer 40. The mixer 40 may be another stirred tank reactor or a static mixer where water is added to promote removal of the water-solubilized products of the esterification reaction, such as the free MCPD isomers.

The water-washed product 109 is subsequently allowed to phase separate in a liquid-liquid separator 50. The liquid-liquid separator 50 may be a settling tank, a three-phase separator, or a disc-stack centrifuge. Regardless of the type of equipment, the liquid-liquid separator 50 provides a treated FOG as a light phase 111 and an aqueous heavy phase 110.

The heavy aqueous phase 110 includes the wash-water and the water-soluble byproducts of the esterification reaction, including the chlorinated species formed during conversion of the organochlorine contaminants. Heavy aqueous phase 110 also includes unreacted polyols.

The light phase 111 is the treated FOG with reduced organochlorine concentrations and ester species. The light phase 111 has an organochlorine concentration less than 5 wppm, and a FFA content less than 5 wt. %.

In some embodiments, light phase 111 may be slurried or contacted with one or more of an absorbent filter media (e.g., diatomaceous earth, cellulose) or adsorbent filter media (e.g., amorphous silica, bleaching clay, ion exchange resin) and is subjected to a filtration step to remove the used filter media, thereby further reducing the metals and phosphorus content if the light phase 111. In embodiments, the contacting and filtration steps are conducted at temperatures between 70° C. and 130° C. and pressures ranging from 100 mbar to 2 bar for about 5 minutes to 1 hour.

The light phase 111 is subsequently hydrodeoxygenated in HDO reactor system 60 where it is brought into contact with a hydrogen-rich treat gas 112. The HDO reactor system comprises a fixed-bed HDO reactor containing sulfided NiMo catalyst. The reactor operates at a temperature range between 290° C. and 370° C. under a pressure between 500 and 2000 psig. The corresponding liquid hourly space velocity for HDO is between 0.3 and 5 h⁻¹, while the hydrogen-to-feed (the light phase 11) ratio is maintained at a value in the 800-1600 NL/L range.

The HDO product is separated from the unreacted hydrogen gas and the gas phase byproducts (including CO, CO₂, H₂S, NH₃, propane, and water vapor in a hot separator (not shown) and the water condensed in a cold separator (not shown). Both the hot separator and the cold separator operate at the reactor exit pressure (minus line losses). The hot separator operates at a temperature between 200° C. and 300° C., whereas the cold separator is operated between 20° C. and 50° C. The heavier hydrocarbons are recovered in the hot separator, and the reaction products of fatty acid hydrogenation and deoxygenation—mainly C15-C18 n-paraffins. In embodiments, a portion of this stream is recycled to the HDO reactor as a reaction solvent or diluent. Propane and C5/C6 hydrocarbons correspond to the polyol portion of the ester. Specifically, propane corresponds to glycerol and C5/C6 corresponds to xylitol/sorbitol. These are mostly condensed in the cold separator as a light hydrocarbon solution. In the case of propane, the fraction remaining in the cold separator gas may be recovered through a membrane and/or by absorption in a hydrocarbon liquid. In embodiments the C5/C6 hydrocarbon cut is used as absorption solvent (in a gas absorption column, not shown, where a propane-containing H₂ bleed gas comes in counter-current contact with C5/C6 hydrocarbons to transfer the propane into the C5/C6 hydrocarbons). This allows the C5/C6 hydrocarbon stream to carry the propane to a product fractionation system 70.

In some embodiments, the hot separator and the cold separator hydrocarbon liquids (with the cold separator liquids including propane and C5/C6 hydrocarbons) from the HDO reactor system 60 are combined to provide a mixed hydrocarbon 113. The mixed hydrocarbon 113 is processed through a fractionation system 70 where it is separated into three hydrocarbon product fractions: A propane fraction 114, a C5/C6 fraction 115, and a heavy hydrocarbon fraction 116.

In some embodiments, the propane fraction 116 is used as a direct replacement of or a supplement to fossil fuel propane for heating, cooking, and transport. The C5/C6 fraction 115 is isomerized into motor gasoline using commercial petroleum refining technologies known to persons skilled in the art. The C5/C6 isomerization technologies include the UOP PENEX and the Axens ATIS-2L processes. With octane numbers reported to be as high as 92, the isomerized C5/C6 fraction may be used as a direct replacement of or supplement to petroleum gasoline.

In some embodiments where the only polyol added for esterification is sorbitol, a pure n-hexane fraction is recovered instead of mixed C5/C6. In such embodiments, the n-hexane may be used for a number of solvent applications such as oil seed extraction. Example of such oil seeds include but are not limited to rapeseed, canola, sunflower, and soybean.

The heavy hydrocarbon fraction 116 comprises the aforementioned C15-C18 n-paraffins. This fraction falls fully in the diesel boiling range and could be used as a compression ignition fuel blendstock. The heavy hydrocarbon fraction 116 may also be hydrocracked/isomerized according to methods disclosed in the prior art (e.g. U.S. Pat. Nos. 7,846,323, 7,968,757, and 8,558,042) to provide a drop-in RD or for sustainable aviation fuel.

### EXAMPLES

### Example 1

A continuous esterification system was operated according to the conditions described in the specifications herein. An esterification reaction process using six CSTRs in series was operated with increasing temperature in a range from about 175-250° C. and at a pressure of about 345-375 mbar. FOG feedstock containing a blend of feedstocks including Used Cooking Oil and Distillers Corn Oil, and had an FFA content of about 20 wt. %, was preheated and fed into the first reactor. Technical glycerol was added along with the FOG feedstock at about 15 wt. % (on a FOG feedstock basis). The total residence time in the six reactors was approximately four hours.

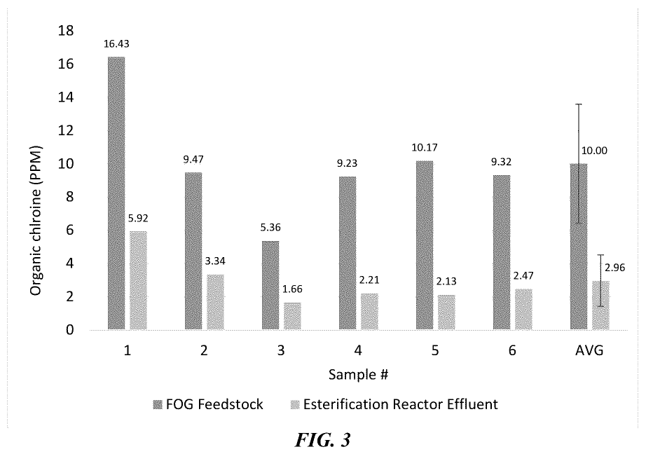

Samples were taken from sampling locations before and after the esterification system to measure the organochlorine content of the FOG feedstock and the esterification reactor effluent, respectively. Six samples were taken from each sampling location at approximately eight hour intervals over a period of about two days for a total of 12 samples. Each sample was subsequently analyzed for organochlorine content on an XOS Clora by monochromatic wavelength dispersive X-ray fluorescence in accordance with ASTM D4929. The results of this analysis are summarized in FIG. 3. On average, the FOG feedstock had an organochlorine content of about 10 ppm whereas the esterification reactor effluent had an average organochlorine content of about 3 ppm; a reduction of approximately 70%.

### Example 2

FOG feedstock was subjected to a continuous esterification process in the same or similar manner as disclosed in Example 1. The FOG feedstock was a blend of feedstocks including Distillers Corn Oil, Used Cooking Oil, and Brown Grease, and had an average FFA content of about 21 wt. %. The FOG feedstock organochlorine content was about 17 ppm and was reduced to 10 ppm following the esterification reaction; a reduction of approximately 40%.

### Example 3

A batch esterification reaction was conducted using a 1 L stirred round-bottom flask heated to about 235° C. FOG feedstock was fed to the reaction vessel and heated up to the reaction temperature and at a pressure of about 20 mbar. Once water and air were fully removed from the FOG feedstock, technical glycerin was added to the reaction vessel at a dose rate of about 12 wt. % on a feedstock basis. A magnetic stir bar was used to mix the reactor contents at about 300 rpm. The reaction progressed isothermally for approximately five hours, during which all vapors and gases were directed out through the top port of the reaction vessel and through a condenser operating at about 60° C. All water vapor and any other condensable materials were collected from the condenser in a round bottom flask and any non-condensable gases were exhausted to a fume hood.

The FOG feedstock used in this experiment was primarily Used Cooking Oil. The FFA content prior to the esterification reaction was about 12 wt. % and the organochlorine content was about 22 ppm. Following the reaction, the esterified reaction product had an FFA of less than 0.1 wt. % and an organochloride content of about 16 ppm and an organochloride reduction of about 26%.

It will thus be seen according to the present invention a highly advantageous method for the removal of organochlorines from fats, oils, and greases has been provided. While the invention has been described in connection with what is presently considered to be the most practical and preferred embodiment, it will be apparent to those of ordinary skill in the art that the invention is not to be limited to the disclosed embodiment, and that many modifications and equivalent arrangements may be made thereof within the scope of the invention, which scope is to be accorded the broadest interpretation of the appended claims so as to encompass all equivalent structures and products.

The inventors hereby state their intent to rely on the Doctrine of Equivalents to determine and assess the reasonably fair scope of their invention as it pertains to any apparatus, system, method or article not materially departing from but outside the literal scope of the invention as set out in the following claims.

1. A method for treating a FOG feedstock for hydrodeoxygenation comprising the steps of:
(a) mixing the FOG feedstock and a polyol in an esterification reactor to provide an esterification product;
(b) washing the esterification product with water to provide a water-washed product; and
(c) separating a treated FOG light phase from the water-washed product, wherein the FOG feedstock has an organochlorine content between 10 wppm and 50 wppm and the treated FOG light phase has an organochlorine content less than 5 wppm.

2. The method of claim 1, wherein the FOG feedstock has an FFA content greater than 5 wt. %.

3. The method of claim 1, wherein the FOG feedstock has an FFA content greater than 10 wt. %.

4. The method of claim 1, wherein the FOG feedstock has an FFA content greater than 12 wt. %.

5. The method of claim 1, wherein the esterification reactor operates at a temperature between 150° C. and 280° C.

6. The method of claim 1, wherein the esterification reactor operates at a pressure between 0.1 psia and 7 psia.

7. The method of claim 1, wherein the FOG feedstock is contacted with water before mixing with the polyol.

8. The method of claim 1, wherein the polyol is glycerol.

9. The method of claim 1, wherein the polyol is xylitol and/or sorbitol.

10. The method of claim 1, further comprising subjecting the treated FOG light phase to a hydrodeoxygenation process by contacting the treated FOG light phase with a hydrogen-rich treat gas to produce mixed hydrocarbons.

11. The method of claim 10, wherein the mixed hydrocarbons comprise C5/C6 hydrocarbons for motor gasoline.

12. The method of claim 10, wherein the mixed hydrocarbons comprise n-hexane.

13. A treated FOG composition for hydrodeoxygenation, comprising;
(a) fatty acid esters of sorbitol or xylitol or erythritol;
(b) fatty acid esters of glycerol;
(c) organochlorine concentration between 1 wppm and 5 wppm; and
(d) free fatty acid concentration less than 5 wt. %.

14. The composition of claim 13, wherein the composition is produced by esterification of a FOG feedstock with a polyol comprising xylitol or sorbitol.

15. The composition of claim 14, wherein the esterification is conducted at a temperature between 150° C. and 280° C.

16. The composition of claim 14, wherein the esterification is conducted at a pressure between 0.1 and 7 psia.

17. The composition of claim 13, wherein the FOG feedstock has an organic chlorine content between 10 wppm and 50 wppm.

18. A method for producing mixed hydrocarbons from a FOG feedstock, said method comprising the steps of:
(a) mixing the FOG feedstock and a polyol in an esterification reactor to provide an esterification product;
(b) washing the esterification product with water to provide a water-washed product;
(c) separating a treated FOG light phase from the water-washed product, wherein the FOG feedstock has an organochlorine content between 10 wppm and 50 wppm and the treated FOG light phase has an organochlorine content less than 5 wppm; and
(d) subjecting the treated FOG light phase to a hydrodeoxygenation process by contacting the treated FOG light phase with a hydrogen-rich treat gas to produce mixed hydrocarbons.

19. The method of claim 18 further comprising separating the mixed hydrocarbons into a propane fraction, a C5/C6 fraction, and a heavy hydrocarbon fraction.

20. The method of claim 18, wherein the hydrodeoxygenation process produces hydrocarbons in one of a diesel boiling range and a gasoline boiling range.
